SEQUENCE LISTING (33 sequences)

SEQ ID NO: 1 - DNA, 301 bp, Unknown
  Bovinae
gcccttctcccagcagaccacctgatgagactgttctgttgcaggaatcattgcaaaagaccccacgtcactagaggaggagatcaaagaaatccgccggagcgggagcagtaaggccctggacaacactcccgagtttgagctctcggayattttctatttctgccggaaaggaatggagaccatcatggacgatgaggtgaccaagaggttctcggcagaggagctggagtcctggaacctgctgagcaggaccaattataacttccagtacatcagcctgcggctcaccgtgctgtgg

SEQ ID NO: 2 - DNA, 301 bp, Unknown
  Bovinae
gtctctagtctatcccttccctgggcccatccctaacagcctcccacaaaacatccctcctcttactcaaacccctgtggtggtgccgcctttccttcagcctgaagtaatgggagtctccaaagtgaaggaggctatggctcctaagcamaaagaaatgcccttccctaaatatccagttgagccctttactgaaagccagagcctgactctcactgatgttgaaaatctgcaccttcctctgcctctgctccagtcttggatgcaccagcctcaccagcctcttcctccaactgtcatg

SEQ ID NO: 3 - DNA, 301 bp, Unknown
  Bovinae
agtcctgccaagcccagccaactaccatggcacgtcacccacacccacatttatcatttatggccattccaccaaagaaaaatcaggataaaacagaaatccctaccatcaataccattgctagtggtgagcctacaagtacacctaccaycgaagcagtagagagcactgtagctactctagaagcttctccagaagttattgagagcccacctgagatcaacacagtccaagttacttcaactgcggtctaaatactctaaggagacatcaaagaagacaacgcaggtaaataagcaaa

SEQ ID NO: 4 - DNA, 301 bp, Unknown
  Bovinae
tttatggccattccaccaaagaaaaatcaggataaaacagaaatccctaccatcaataccattgctagtggtgagcctacaagtacacctaccaccgaagcagtagagagcactgtagctactctagaagmttctccagaagttattgagrgcccacctgagatcaacacagtccaagttacttcaactgcggtctaaatactctaaggagacatcaaagaagacaacgcaggtaaataagcaaaatgaataacagccaagattcatggacttattaataaaatcgtaacatctaaactag

SEQ ID NO: 5 - DNA, 301 bp, Unknown
  Bovinae
gcatgttaggtggagaaggacccaacctcagtgaggccaagtgagaaccacacagcaaaccacgagaagaaatgctgccatggggacccatacaaagatcatttccagccccctgggcaggatggccaagtgcactttccaagacgaagamatcaacctggccagtggcttaggccagccacaaagctgagagctgtctccagaggcctctgtctgtccatctgtttgtttgccctggggaggggtggtcctctcccagactcccacccaatcctgccggcctccactctgactcttccct

SEQ ID NO: 6 - DNA, 301 bp, Unknown
  Bovinae
  unsure (247)..(247): exact sequence at this location unsure
tctttcccttaattaggagaaatttggaaaaaacaagtctcggagcttccagctctttggctctccacccggccagagggacctgctgttcaaagactctgctcttgggtttttgaggatcccctcgaaggtagattcggcgctgtacctkggctcccgctacttgaccaccttgaagaacctcagggaaactgcggaggaggtgaaggcgcggtacaccagggtcgtgtggtgtgccgtgggaccngaggagcagaagaagtgccagcagtggagccagcagagcggccagaacgtgacc

SEQ ID NO: 7 - DNA, 301 bp, Unknown
  Bovinae
ctagagcccataacaatcccaggatcccagagccagagccagcgttttgctgccctctgtgggcggagatagacttgcagtttcccaggagaatgcaggaggggtgggggggggcctgagaccccttgtgtctctctttcggggttcagaytctgcagtggagctgtgggaaccagatgcacaagatgcaagcagccagcccttgggaagcagcaagtgcatcctcagggaggagtccagcacgccgcagtccgctggggacacttcgaggatggggctggaggcgccagagcccacggcc

SEQ ID NO: 8 - DNA, 301 bp, Unknown
  Bovinae
aggctccactagacatcgctgtaaagccatggagaagatgtcaaagttctttttgagttctttagttttgtgactctaatctaaaaagtccacatccactcattcatcgacacagaaatgttactccccacaggaattcatccagcaaacrattgcccattccagtttccccttctggatggggttgtcaatgaggaaacccaattactcgtggctttgggaagatggtactcctttgacgccccacttgtaagtttgatgctcttttttagttttgtttatttatttatttctatctttg

SEQ ID NO: 9 - DNA, 301 bp, Unknown
  Bovinae
tgccaaaaaatgtcatctgctctcaataaattgagagcaggcgaggggaggtgtaactggataaggggtaggttttaattttttaacaggcagaaggaggaggaggataccatcatggttagtcagtcagcaaggattaatcctcactaaygtctcccattcaaacccattcttctctccacagctgtgcaaacataaattaattgggtcattttgatgaaggcatataagaaaaaaattttaatgtaggcaaaggtagtaattagataaataacacattctctaaatgcttaggaaggac

SEQ ID NO: 10 - DNA, 301 bp, Unknown
  Bovinae
ttttgatacatggaaaattttggcccatcagattgttctgtgacagccagtctcagttctaaaaggttacctgacatttgcttttctgaagtagaagtcagtgtagcttagtataaataggatcttttttttccaccttcctgggagcagyggaaacaaagataatgcaaagccaaaggaaagaaccagggacttgcaaataatttaaatagaaataagcaaatttcaaaaaagtgtgagttttccatcattgtttttctcttaaaagacttccatagcagtccagtggttaagacttcat

SEQ ID NO: 11 - DNA, 301 bp, Unknown
  Bovinae
ctccgcagtcttaggtgtgtggctgccttgttcttcttcagatgaaggcattccaagagctcactgggtctgtctgtggagagagaccaagccgcttctgtggtcacaggtacgccaggcaggcagacctattcctgccagagccttagtytgctcagcagctgtttgaatggaattatttgcgaagggaaaggagacaggtaatttctctaagctcaagttttattcctgagttagctagcagcttcgcctctcagttgtgtgcgtggatctgctctggtacagtgttggtcttttgctt

SEQ ID NO: 12 - DNA, 301 bp, Unknown
  Bovinae
tgagaagttggcggagattatctggcagaaccggcagcagatccgcagagccgagcacctctgccagcagctgcccatccccggccccgtggaggagatgctggctgaggtcaacgccaccatcactgacatcatctcagccctggtgacsaggtgactcctggccacgccccgctcccatctggttgccctgggttgggggcagcagggtctttgcagatggggagctctggcttaaatccttcagtttctgcctcacaccctcctcccatccctctccatcccctgttgctatggcctc

SEQ ID NO: 13 - DNA, 301 bp, Unknown
  Bovinae
tggcctgaagactgagcccgccccgtctgcctgcagcctgtctcctccagggaggccctcggggcccccttcagctctcaccctctgcccagtctccgttccagccgtcgagggatgcgaacttgggaatggagttggccctccccagccyggtgctgacggagaggaccatctgaggccagctttgttcgcggggaggagggcgctggcctggcctgcatccccaccatgttcctgctgctgcctttcgacagcctgattgtcagcctcctgggcatctccctgacggtcctcttcaccc

SEQ ID NO: 14 - DNA, 301 bp, Unknown
  Bovinae
cacccagggctattcctgtctggtccctgcctctaacacattctgtatcccagacttttccacagaactgagagtgctgtgcttcttcagttgctctaaccacacgaatagggcaggcgcgatgttgcccgggacagtggcccaagcctcytttggatggaaggaactgaattaaggaattcccgccagaggaccaggaagcctgaggtcatgtttctgtcatttgcccattcatcaaacagtcaccaagtgaccagggccactgggttaaatgctagttgaaaatcataaagagaaatta

SEQ ID NO: 15 - DNA, 301 bp, Unknown
  Bovinae
tggaggggccagtggtcgtgcctgaccttgcgttgtcctagaggggtcagagcccatgcttgtctctgccagggaaacccagggaccacgactcggcggagacgggcggcgacagcttcgagagctcggagtcgctgctgcagtcctggamcagccagtcgtctctgctggacgtgcagcgggtgccctccttcgagagcttcgaggacgactgcagccagtccctgggcctcagcaagccgaccatgtccttcaaggactacatccaggaccggagtgacccagtggagcagggcaaacc

SEQ ID NO: 16 - DNA, 301 bp, Unknown
  Bovinae
  unsure (151)..(151): exact sequence at this location unsure
tcctctgtcaggtgtactatttaaaatacacttgacccttgaacaaaatgggtttaagctgcgcaggtctgctcacactacagatttttttcaatagcagatactgcaatactacatgatkcgwgattggttgaatccacagatacaaagnaaccacaaatacagaaggccaattataaattatactcaggttttcaactgttgcagagtcagtcagcacccctgactcctgccttcttcaagggtcagatacatgtagccgtgtctagtgattataaacgaattcatatttctgtaaaga

SEQ ID NO: 17 - DNA, 298 bp, Unknown
  Bovinae
actttattaacaaaactgcttaaatttcctctgtcaggtgtactatttaaaatacacttgacccttgaacaaaatgggtttaagctgcgcaggtctgctcacactacagatttttttcaatagcagatactgcaatactacatgatkcgagattggttgaatccacagatacaaaggaaccacaaatacagaaggccaattataaattatactcaggttttcaactgttgcagagtcagtcagcacccctgactcctgccttcttcaagggtcagatacatgtagccgtgtctagtga

SEQ ID NO: 18 - DNA, 301 bp, Unknown
  Bovinae
attctttaccgactgagctacaagggaagcctaccagcttgcctaccagcaatgcctaccagcaaatgttagacatcagagtagaaggtaaaacttggaaatactggttcacaaacctggaaatactggttcacaaaccatgggccttctyattcttcagagaaatccacagttagaaccagaggtggtcttccagatgtaacaccctgcttaccccctcccacattgtctttggatggtcaggaagggcggcagtgaggagacacccctcgtccaaggtatccagatgtaacaccctgct

SEQ ID NO: 19 - DNA, 301 bp, Unknown
  Bovinae
ttgcttgtgaatctaaaacacactagtccagcaaataaaacaaaccaccctctaattaacctcctatccaatttttccttttcagggattacaggaggaaaagattttagagacattgaaagtaaatttgctctcaggactcctgaagacwcagctgaggacacttgccacctcattcctggagtgaccgaatctgtggctaactgtcacttcaaccacagcagcaaaacctttgtggtgatccatggctggacggtaagggaggctctttaggaaggagtagactggcatgaactaagcg

SEQ ID NO: 20 - DNA, 301 bp, Unknown
  Bovinae
tggctcttcctctgctggaggcttcaggcggactcggggtgaacggggaggctgcactgttgttacagcctgttgaccgctgtgtccccgggcagctgcggaggaggtgaaggcgcggtacaccagggtcgtgtggtgtgccgtgggaccygaggagcagaagaagtgccagcagtggagccagcagagcggccagaacgtgacctgtgccacggcgtccaccactgacgactgcatcgtcctggtgctggtagggggcctgctgccggcctgcaagccgtgcgggtggggggcacaggtc

SEQ ID NO: 21 - DNA, 301 bp, Unknown
  Bovinae
agtgttgcccttggctcccagggagagcatcgcaaaggctgaagcgatgctcacgggggagaagaagatgttgctggtgttggactgatgagccaaatggtggtatatgctgaaggcaaagttggccaggttgggggcaatcttgtggcaygctgcttcctggtgggatgtatcatctgtctcttggacagcgtgtccttggagaactccagccagggagatgggggccaggcagcacagggctgccagcagcagaaggccccgcgtgatggagagtgccattatcctgcaagagagaagg

SEQ ID NO: 22 - DNA, 301 bp, Unknown
  Bovinae
gtaattatcaggcatgattttcagaataaactgttttcttcagtcaccacaagctttactagaaaactgaaacagtgggtgaatgataagacaacttatcatattactgctaattggggccatttttcattaaaatggcattattgccccyaagacatcagtatacaaaattattcagaatgtaatgaattgagtatcctggaagagcagcatggattcaaaagtatactttgagtaaagacaactctcacacaattttaccatattggaggacaataaagatgttcattacccaggaatg

SEQ ID NO: 23 - DNA, 301 bp, Unknown
  Bovinae
gagcaaaatgaatcataaactagggagggggcagggttttggactacattttgtcatctactcgagggataagaggcacggagggargcgaacgtgcatcattacctgggccgccggctatggggcgatcttgaacgtgacgcgcacaggrgcgaatttcggtgtgtgcggtgtctgcagtggcctgactcatagtagtagcaagatctgagagagaaggaaaaacaaaaagggcatttgcaactgccagcaaaacycagtttatcagcatgactgagtagcccctggaattctgtcttaa

SEQ ID NO: 24 - DNA, 301 bp, Unknown
  Bovinae
ccttgaaaaccactgtcacttccccagtatgaactccctaaagttaggggtgagttttgtgttcattgtagaagagagggcaacgtatgttggaggattcattttccagccctcttcacatccctcctgatttctcttgaagtgataaacmtttcggtatctaacttgactaattctatatccttgacatttaaactccccatcccactgtttctcaatctggggacgaaagatataactttacatacatgttaaaatcaaaagacttatgtgaaaatgcacattttaccacagagatata

SEQ ID NO: 25 - DNA, 301 bp, Unknown
  Bovinae
ccttaccagtcttgcttttctagccactgtgccagatattgtctgatttccatgggaaaactgtcatcatagagctggtgaacctgctccaggtatttggactcaagctgctgaagctcataccactgggacatcctatagggaaagagasagcacggatcataaatggaagcccacagctccaaagaaagcatgtctagcattgtgcttcttctaaatatcttgcttaatcaaaaacataatttgtctcaaatatacttttcaaatggatgtctatattccaaaacctgttttgttttgt

SEQ ID NO: 26 - DNA, 301 bp, Unknown
  Bovinae
  unsure (291)..(291): exact sequence at this location unsure
caatttctgccatacccatattatgcaaagccagctgcagttaggtcacctgcccaaattcttcaatggcaagttttgtcaaatactgtgcctgccaagtcctgccaagcccagccaactaccatggcacgtcacccacacccacatttakcatttatggccattccaccaaagaaaaatcaggataaaacagaaatccctaccatcaataccattgctagtggtgagcctacaagtacacctaccaccgaagcagtagagagcactgtagctactctagaagcttctccngaagttattg

SEQ ID NO: 27 - DNA, 287 bp, Unknown
  Bovinae
tattaacaaaactgcttaaatttcctctgtcaggtgtactatttaaaatacacttgacccttgaacaaaatgggtttaagctgcgcaggtctgctcacactacagatttttttcaatagcagatactgcaatactacatgatgcgwgattggttgaatccacagatacaaaggaaccacaaatacagaaggccaattataaattatactcaggttttcaactgttgcagagtcagtcagcacccctgactcctgccttcttcaagggtcagatacatgtagccgtgt

SEQ ID NO: 28 - DNA, 301 bp, Unknown
  Bovinae
atgccctttttgtttttccttctctctcagatcttgctactactatgagtcaggccactgcagacaccgcacacaccgaaattcgcccctgtgcgcgtcacgttcaagatcgccccatagccggcggcccaggtaatgatgcacgttcgcytccctccgtgcctcttatccctcgagtagatgacaaaatgtagtccaaaaccctgccccctccctagtttatgattcattttgctcgtgcaaatggaggtggttgacttaatggtgaagttatgtttctcaggtatgacagctacgagga

SEQ ID NO: 29 - DNA, 301 bp, Unknown
  Bovinae
gcagagagggtgaaatggtgtgaacagggtatgtagtgtgacctacatactggcttctctgctagtgtatctcctaaaggaaaagtttccttataacacaagtcagagtgacttctgttttccttggccctccctagactcattttactcygaagcaaactcagcttaagattttcaacttatatccaggacaaaaataccttgtgcagattcgctgcaagccagaccatggatactggagtgagtggagcccagagagctccatccagatacctaatggtaagtgtcttgagattcccca

SEQ ID NO: 30 - DNA, 301 bp, Unknown
  Bovinae
tctacagtttcatatctaccactttacttcatacaaccagcatgtttggagtggtgcatcaataagacaaatcgcagagtatttcctgaattatttatactctctctgttagtttatattgaatatactgggtttatatggtggtttcatrcaaacttagctgatgattatttaaaatcctttccctactctttctgagttatagaaatatttttctttccctctgagaataaaatttcaagtgcattctttggtgtgtaatcatcatttccatcgttcatactggcccatgaggctgtgg

SEQ ID NO: 31 - DNA, 301 bp, Unknown
  Bovinae
gcccggcggcctggcctgctagccccgccctcccttccagatctctactacttcctcttcgcccccaccctgtgctacgagctcaacttcccccgctccccccgcatccgaaagcgcttcctgctgcggcgactcctggagatggtgaggsggggcctcgcgggccagggtgggcgggcctgccggcacccggcaccggggctcagctcactgtccgcttgcttccttccccagctgttcctcacccagctccaggtggggctgatccagcaggtacgtgcccgggggggggggggggggg

SEQ ID NO: 32 - DNA, 301 bp, Unknown
  Bovinae
agcaagatcctttctctgtgatgcaacctctcaggttgatgcaatccctttgccatcgcccacactgtttattgttctttgtacttcgttttctcttatgccacaccctacccacaatcctgtccttgggtccaacgccactgaaatccayagttgtgatgaccctttggggtccctctaaagcaaaagtgtccctttgtctcttgatgcttaattctgctgccaacacccacacgttctctctctagatttatcttgggcttgtcgtcttcctccttgttcaaactctctctacccctgc

SEQ ID NO: 33 - DNA, 301 bp, Unknown
  Bovinae
aagacacacaccaaggcccagggtcacaagtgcccaacctctggcccttaattctggcttctggagaagtcaccatcccaaaggcctccttctaatgggatctgggaactcctattcactgggaaaagtcaagggcaggtgatgttcctgycgacaccatcagtgggcactcaggagactagctcctggcccagtgtgtgcatgctaagtcacttcagttgtgtctgactctttagaccccatggactgtagcccaccagggtcctttgtccatggggattctccaggcaagaatattggc

(19) United States
(12) Patent Application Publication
(10) Pub. No.: US 2026/0234734 A1
(43) Pub. Date: Aug. 13, 2026

(54) Method for Predicting the Milk Clotting Ability Index and Gene Chip Adapted to Carry Out this Method

(71) Applicants: Universita Degli Studi Di Padova, Padova (IT); Intermizoo S.P.A., Padova (IT)

(72) Inventors: Martino Cassandro, Dolo (IT); Filippo Cendron, Paese (IT); Francesco Cobalchini, Dueville (IT); Francesco Veronese, Boschi Sant’Anna (IT)

(21) Appl. No.: 18/548,284

(22) PCT Filed: Feb. 28, 2022
(86) PCT No.: PCT/IB2022/051745

§ 371 Date: Aug. 29, 2023

(30) Foreign Application Priority Data
Mar. 1, 2021 (IT) 102021000004640

(51) Int. Cl. C12Q 1/6888

(52) U.S. Cl. CPC C12Q 1/6888; C12Q 2600/124; C12Q 2600/156

(57) ABSTRACT

A gene chip for predicting the milk clotting ability index (IAC) of the milk produced by a bovine animal, includes, as probes locked onto the surface of such gene chip, at least fourteen DNA nucleotide sequences different from each other, selected from among the DNA nucleotide sequences having at least 90% identity with the fourteen SEQ. ID. NO. 1 to 12, 32 and 33. The gene chip is configured to simultaneously identify the presence or absence in a biological sample of the bovine animal of each genotype at the gene locus corresponding to at least fourteen homozygous or heterozygous single nucleotide polymorphisms (SNP) comprised in the group of polymorphisms (SNP) constituted by SNP rs109913786 (SEQ. ID. NO. 1); SNP rs43703012 (SEQ. ID. NO. 2); SNP rs43703015 (SEQ. ID. NO. 3); SNP rs43703017 (SEQ. ID. NO. 4); SNP rs110137537 (SEQ. ID. NO. 5); SNP rs43765462 (SEQ. ID. NO. 6); SNP rs134390757 (SEQ. ID. NO. 7); SNP rs135588030 (SEQ. ID. NO. 8); SNP rs41624917 (SEQ. ID. NO. 9); SNP rs109007595 (SEQ. ID. NO. 10); SNP rs109578101 (SEQ. ID. NO. 11); SNP rs137182814 (SEQ. ID. NO. 12) (STAT5A); SNP rs109975461 (SEQ. ID. NO. 32); SNP rs134589272 (SEQ. ID. NO. 33).

### INCORPORATION BY REFERENCE OF SEQUENCE LISTING

This application includes a Sequence Listing submitted electronically in ASCII format. The ASCII copy of the Sequence Listing, created on Jul. 8, 2025, is named 19720-130-ReplacementSeqList2.txt and is 18,985 bytes in size. The ASCII copy of the Sequence Listing is expressly incorporated herein by this reference.

### BACKGROUND OF THE INVENTION

### 1. The Field of the Invention

The present invention relates to a method for predicting the milk clotting ability index (IAC) of the milk of a bovine animal, and to a gene chip adapted to carry out said method.

### BACKGROUND

Milk clotting ability is a characteristic defined as the coagulation capacity of milk to react with a coagulating compound to form a curd within an optimal time frame that is also of a suitable consistency for further processing.

Milk is known to have a high processing efficiency if it coagulates quickly and the resulting curd has a high consistency. Several studies in the 1980s (Pecorari et al., 1987) showed that the cheese yield was inversely proportional to the decrease in coagulation time and directly proportional to the increase in curd consistency.

Furthermore, these studies have shown that milk with a sub-optimal milk clotting ability has a low cheese yield, which may affect the final quality of the cheese, its composition, the incidence of defects and rejects, and the processing time and cost.

To date, milk clotting ability is measured using a lactodynamograph.

The lactodynamograph is used to simulate a cheese-making process in which the milk passes from a solution to a gel.

To do so, a sample of milk is brought to a temperature of 35° C. and a standard amount of rennet is added. The behaviour of the milk sample is then monitored by an instrument for a period of about 30 minutes.

The change in the consistency of the milk is then measured and reproduced in graphs called lactodynamograms, from which two fundamental parameters can be derived to calculate the milk clotting ability index (IAC) and thus recognise the quality of the milk:
R, coagulation time (expressed in minutes): it represents the time between the addition of rennet and the start of the coagulation process, i.e., the moment when a change in the viscosity of the sample is detected;
A30, curd consistency: it is measured in mm and corresponds to the lot amplitude reached 30 minutes after the rennet is added.

The milk clotting ability index (IAC) combines these two main milk clotting ability properties, R and A30, expressed as an index on a 100 basis with a weight of 50% each.

Milk samples obtaining an IAC value of more than 100 are more suitable for the cheese-making process than milk samples with IAC values below 100.

The IAC formula is as follows:

IAC=100+[((A30−average_{A30})/dS_{A30})×2,5−((R−average_{R})/dsR)×2,5]
wherein
IAC is the milk clotting ability index;
A30 is the consistency of the curd (in millimetres);
R is the coagulation time (in minutes);
ds is the standard deviation.

By calculating this IAC, it is therefore possible to distinguish between milk that is optimal for cheese making and milk that is not.

Choosing milk with a high IAC for cheese making can result in a yield increase of up to 10% compared to milk with a low IAC.

Disadvantageously, the measurement of milk clotting ability and the IAC by means of a lactodynamograph is very costly and time-consuming and therefore, from a practical point of view, it is basically impossible to monitor an entire bovine population with this standard technique.

Recently, a new method for calculating IAC using near- and mid-infrared spectroscopy (NIR-MIR) has been identified (De Marchi et al., 2009) for milk analysis.

This method is based on the ability of each biological substance to absorb light in the mid-infrared region, producing its own characteristic absorption spectrum, from which the IAC can be predicted to a good approximation.

It is clear that, with both these methods, standard and spectroscopic, the IAC is only detectable from the analysis of a milk sample.

The milk is produced when the cow is already an adult and the milk has been milked, i.e., the farmer has already spent time, equipment and materials to obtain this milk.

If the IAC of the milk is not optimal and the milk does not have good coagulation properties, the farmer has to decide whether to leave the liquid form of the milk unaltered and thus not to use it for cheese making, or to use the milk for cheese production, even if the coagulation performance is lower. Both options will, however, result in economic losses.

Also known is the scientific publication Viale et al.: “Association of candidate gene polymorphisms with milk technological traits, yield, composition, and somatic cell score in Italian Holstein-Friesian sires”, Journal of Dairy Science, vol. 100, no. 9, September 2017 (2017-09), pages 7271-7281. This publication describes the genotyping of 96 polymorphisms SNP to assess the influence on milk coagulation properties (MCP), including rennet coagulation time (RCT) and curd compactness (a30).

Also known is the scientific publication Beux Simone et al: “Effect of THI on milk coagulation properties of Holstein-Friesian dairy cattle”, Revista Brasileira de Zootecnia, vol. 46, no. 5, May 2017 (2017-05), pages 429-432.

Also known is the scientific publication Pegolo S. et al.: “Effects of candidate gene polymorphisms on the detailed fatty acids profile determined by gas chromatography in bovine milk”, Journal of Dairy Science, vol. 99, no. 6, June 2016 (2016-06), pages 4558-457 and corresponding Supplemental Data. This publication describes the genotyping of 96 polymorphisms SNP using the Illumina GoldenGate system.

### DESCRIPTION OF THE INVENTION

It is the purpose of the present invention to provide a method for predicting the milk clotting ability index (IAC) of milk that overcomes the limitations of known methods for calculating IAC.

It is also an object of the present invention that such a method enables the prediction of the IAC before the milk is produced.

It is also an object of the present invention that the above-mentioned method allows the calculation of the IAC for a large number of samples.

It is still an object of the present invention to make a device which is adapted to carry out the above method in a direct and simple manner.

The above purposes are achieved by a gene chip, as disclosed herein.

The purposes are further achieved by a method for predicting the IAC of milk produced by a bovine animal, as disclosed herein.

Further characteristics of the method and chip are provided in the dependent claims.

Other aspects and advantages of the chip and method of the invention will be apparent to a person skilled in the art from the following description of a preferred embodiment of the invention which is given by way of non-limiting example.

### DETAILED DESCRIPTION OF THE INVENTION

The present invention relates to a method for predicting the milk clotting ability index (IAC) of milk produced by a bovine animal, comprising a step a) which includes:
a) determining in a biological sample of the bovine animal the genotype at the gene locus corresponding to at least fourteen homozygous or heterozygous single nucleotide polymorphisms SNP selected from:
   SNP rs109913786 (SEQ. ID. NO. 1) of the Glycerol-3-phosphate acyltransferase 6 (AGPAT6) gene;
   SNP rs43703012 (SEQ. ID. NO. 2) of the Casein β (CSN2) gene;
   SNP rs43703015 (SEQ. ID. NO. 3) of the Casein kappa (CSN3) gene;
   SNP rs43703017 (SEQ. ID. NO. 4) of the Casein kappa (CSN3) gene;
   SNP rs110137537 (SEQ. ID. NO. 5) of the Hormone-sensitive lipase (LIPE) gene;
   SNP rs43765462 (SEQ. ID. NO. 6) of the Lactotransferrin (LTF) gene;
   SNP rs134390757 (SEQ. ID. NO. 7) of the Oxysterols receptor LXR-α (LXRα) gene;
   SNP rs135588030 (SEQ. ID. NO. 8) of the Oxidized low-density lipoprotein (lectin-like) receptor 1 (ORL1) gene;
   SNP rs41624917 (SEQ. ID. NO. 9) of the Phospholipase C epsilon 1 (PLCE1) gene;
   SNP rs109007595 (SEQ. ID. NO. 10) of the POU class 1 homeobox 1 (POU1F1) gene;
   SNP rs109578101 (SEQ. ID. NO. 11) of the Signal transducer and activator of transcription 5A (STAT5A) gene;
   SNP rs137182814 (SEQ. ID. NO. 12) of the Signal transducer and activator of transcription 5A (STAT5A) gene;
   SNP rs109975461 (SEQ. ID. NO. 32) of the Cholinergic Receptor Nicotinic Epsilon Subunit (CHRNE) gene;
   SNP rs134589272 (SEQ. ID. NO. 33) of the Scribble cell polarity complex component LLGL2 (FDXR LLGL2) gene.

It is noted that the terms polymorphism, single-nucleotide polymorphism and the abbreviation SNP (single-nucleotide polymorphism) will be used interchangeably throughout this document.

The numerical code indicated after the abbreviation “rs” represents the unique identification code assigned by the National Center for Biotechnology Information (NCBI) to a group or cluster of SNPs located at the same gene locus.

In particular, the technical information relating to the SNPs mentioned above and those that will be described later, such as for example their position in the genome and the corresponding nucleotide are variations, publicly available in the database https://www.ncbi.nlm.nih.gov/projects/SNP/snp_summary.cgi.

It has been found that the use of at least fourteen polymorphisms SNP having SEQ. ID. NO. 1 to 12, 32 and 33 as identified above allows for an increase in the accuracy of IAC prediction compared to the prior art.

Moreover, known previous papers, including the scientific publications Viale et al., 2017, Beux Simone et al, 2017 and Pegolo S. et al., 2016, do not describe the use of polymorphisms SNP having SEQ. ID. NO. 32 and 33. These two specific SNPs are known as SNPs associated with mastitis; but in the state of the art they have never been shown to be related to milk clotting ability. In addition, it has been experimentally found, as explained below, that the addition of these two SNPs is able to improve by approximately 2% or more the accuracy of IAC prediction obtained with SNPs having SEQ. ID. NO. 1 to 12.

According to the method of the invention, the biological sample comprises a bovine nucleic acid, preferably DNA.

It is not excluded that, according to an alternative aspect of the invention, the biological sample may comprise RNA or the protein corresponding to the sequenced gene.

Preferably, such a biological sample is a sample of tissue, blood, semen, hair or nasal mucus, from the bovine animal.

Furthermore, it is not excluded that, according to variants of the invention, the biological sample of the bovine animal is of a different type from that indicated provided that it contains at least one bovine nucleic acid.

According to an aspect of the method of the invention, the biological sample is a biological sample of Holstein Friesian cattle.

It is not excluded that, according to alternative embodiments of the invention, the biological sample comes from a bovine animal of a different breed than specified.

According to the method of the invention, the determination of the genotype at the gene locus corresponding to the SNP polymorphisms disclosed above is established by nucleic acid analysis of the biological sample, preferably by DNA analysis.

DNA testing is carried out using any of the methods for identifying homozygous or heterozygous polymorphisms SNP in a genetic sequence.

Some examples not to be considered limiting of such identification methods may include SNP gene chips, such as by the gene chip of the present invention which is defined in detail below, SNPRFLP, dynamic allele specific hybridization (DASH), TaqMan probes, primer extension, oligonucleotide ligation assay, single strand conformation polymorphism, TGGE temperature gradient gel electrophoresis, denaturing gel high performance liquid chromatography, high resolution whole amplicon melting (HRM PCR), SNPlex and/or next generation sequencing (NGS) on the biological sample.

Returning to the method of the present invention, it also comprises a step b), subsequent to step a), comprising:
b) assigning a predefined value V to each of the polymorphisms that were determined in step a) above.

Thereafter, the method of the invention comprises a step c) comprising calculating the arithmetic mean of the predefined values V assigned in step b) to the SNPs that were determined in step a).

Advantageously, this arithmetic mean essentially corresponds to the milk clotting ability index (IAC) of the milk to be produced from the bovine animal to which the above-mentioned biological sample belongs.

In particular, the applicants carried out genomic analysis of more than 4000 bovine animals and correlated the data obtained from this analysis with phenotypic data from the same animals and with the IACs determined in the milk of the above-mentioned sampled animals. Thanks to the processing of such data by the applicants themselves, predefined values V to be assigned to the SNPs defined above have been identified which allow the IAC of the milk to be produced by the said bovine animal to be obtained by means of the method of the invention.

In detail, the above-mentioned predefined value V of the method of the invention is comprised between:
102 and 106 for SNP rs109913786 (SEQ. ID. NO. 1);
86 and 125 for SNP rs43703012 (SEQ. ID. NO. 2);
100 and 110 for SNP rs43703015 (SEQ. ID. NO. 3);
103 and 108 for SNP rs43703017 (SEQ. ID. NO. 4);
102 and 107 for SNP rs110137537 (SEQ. ID. NO. 5);
102 and 108 for SNP rs43765462 (SEQ. ID. NO. 6);
102 and 107 for SNP rs134390757 (SEQ. ID. NO. 7);
105 and 107 for SNP rs135588030 (SEQ. ID. NO. 8);
103 and 106 for SNP rs41624917 (SEQ. ID. NO. 9);
100 and 109 for SNP rs109007595 (SEQ. ID. NO. 10);
102 and 109 for SNP rs109578101 (SEQ. ID. NO. 11);
103 and 107 for SNP rs137182814 (SEQ. ID. NO. 12);
98 and 101 for SNP rs109975461 (SEQ. ID. NO. 32);
98 and 101 for SNP rs134589272 (SEQ. ID. NO. 33).

Advantageously, the method of the present invention makes it possible to predict the IAC of the milk produced by a bovine animal, before the bovine animal effectively produces it.

In fact, the method exploits the genotyping of the bovine animal, which can be carried out at its birth, thus anticipating the waiting time needed to evaluate the bovine animal according to the performance of its productive career or that of its daughters.

It is thus possible to implement the cheese yield by allowing the farmer to select the best performing animals suitable for the production of cheese and dairy products.

Even more advantageously, the presence of a method for selecting the best cattle for dairy production allows the planning of a cheese production process with less waste, a lower environmental impact and greater sustainability of the supply chain.

Further advantageously, the determination of the polymorphisms identified in the present invention, in particular SNPs with SEQ. ID. NO. 1 to 12, 32 and 33, makes it possible not only to predict the IAC of the milk produced by the genotyped cattle, but also to compare the milk clotting ability of a plurality of bovine animals with each other.

The prior art teaches that milk samples having an IAC of more than 100 are more suitable for cheese making than milk samples having IAC values of less than 100; however, with the method of the invention it will also be possible to determine, even among cattle having an IAC of more than 100, which of them is more suitable for cheese making.

In fact, the higher the IAC obtained from the bovine animal according to the method of the invention, the better the dairy processing of the milk produced by such bovine animal.

According to an aspect of the method of the invention:
if the genotype at the gene locus corresponding to the SNP rs109913786 (SEQ. ID. NO. 1) is C/C or T/T, the predefined value V assigned to this SNP is comprised between 103 and 106, preferably about 104;
if the genotype at the gene locus corresponding to the SNP rs43703012 (SEQ. ID. NO. 2) is C/C, the predefined value V assigned to this SNP is comprised between 99 and 102, preferably about 101;
if the genotype at the gene locus corresponding to the SNP rs43703015 (SEQ. ID. NO. 3) is C/C or T/T, the predefined value V assigned to this SNP is comprised between 102 and 109, preferably about 105;
if the genotype at the gene locus corresponding to the SNP rs43703017 (SEQ. ID. NO. 4) is A/A or G/G, the predefined value V assigned to this SNP is comprised between 104 and 106, preferably about 105;
if the genotype at the gene locus corresponding to the SNP rs110137537 (SEQ. ID. NO. 5) is A/A or C/C, the predefined value V assigned to this SNP is comprised between 103 and 106, preferably about 105;
if the genotype at the gene locus corresponding to the SNP rs43765462 (SEQ. ID. NO. 6) is G/G or T/T, the predefined value V assigned to this SNP is comprised between 103 and 107, preferably about 105;
if the genotype at the gene locus corresponding to the SNP rs134390757 (SEQ. ID. NO. 7) is C/C or T/T, the predefined value V assigned to this SNP is comprised between 103 and 106, preferably about 104;
if the genotype at the gene locus corresponding to the SNP rs135588030 (SEQ. ID. NO. 8) is A/A or G/G, the predefined value V assigned to this SNP is comprised between 105 and 107, preferably about 106;
if the genotype at the gene locus corresponding to the SNP rs41624917 (SEQ. ID. NO. 9) is C/C or T/T, the predefined value V assigned to this SNP is comprised between 103 and 105, preferably about 104;
if the genotype at the gene locus corresponding to the SNP rs109007595 (SEQ. ID. NO. 10) is C/C or T/T, the predefined value V assigned to this SNP is comprised between 102 and 107, preferably about 105;
if the genotype at the gene locus corresponding to the SNP rs109578101 (SEQ. ID. NO. 11) is C/C or T/T, the predefined value V assigned to this SNP is comprised between 103 and 108, preferably about 106;
if the genotype at the gene locus corresponding to the SNP rs137182814 (SEQ. ID. NO. 12) is C/C or G/G, the predefined value V assigned to this SNP is comprised between 104 and 106, preferably about 105;
if the genotype at the gene locus corresponding to said SNP rs109975461 (SEQ. ID. NO. 32) is C/C or T/T, the predefined value V assigned to this SNP is comprised between 99 and 101, preferably about 100;
if the genotype at the gene locus corresponding to said SNP rs134589272 (SEQ. ID. NO. 33) is C/C or T/T, the predefined value V assigned to this SNP is comprised between 98 and 101, preferably about 99.
Preferably, also:
if the genotype at the gene locus corresponding to the SNP rs109913786 (SEQ. ID. NO. 1) is C/T, the predefined value V assigned to this SNP is comprised between 103 and 105, preferably about 104;
if the genotype at the gene locus corresponding to the SNP rs43703012 (SEQ. ID. NO. 2) is A/C, the predefined value V assigned to this SNP is comprised between 108 and 111, preferably about 110;
if the genotype at the gene locus corresponding to the SNP rs43703015 (SEQ. ID. NO. 3) is C/T, the predefined value V assigned to this SNP is comprised between 104 and 106, preferably about 105;
if the genotype at the gene locus corresponding to the SNP rs43703017 (SEQ. ID. NO. 4) is A/G, the predefined value V assigned to this SNP is comprised between 104 and 107, preferably about 106;
if the genotype at the gene locus corresponding to the SNP rs110137537 (SEQ. ID. NO. 5) is A/C, the predefined value V assigned to this SNP is comprised between 104 and 106, preferably about 105;
if the genotype at the gene locus corresponding to the SNP rs43765462 (SEQ. ID. NO. 6) is G/T, the predefined value V assigned to this SNP is comprised between 104 and 106, preferably about 105;
if the genotype at the gene locus corresponding to the SNP rs134390757 (SEQ. ID. NO. 7) is C/T, the predefined value V assigned to this SNP is comprised between 103 and 105, preferably about 104;
if the genotype at the gene locus corresponding to the SNP rs135588030 (SEQ. ID. NO. 8) is A/G, the predefined value V assigned to this SNP is comprised between 105 and 107, preferably about 106;
if the genotype at the gene locus corresponding to the SNP rs41624917 (SEQ. ID. NO. 9) is C/T, the predefined value V assigned to this SNP is comprised between 103 and 105, preferably about 104;
if the genotype at the gene locus corresponding to the SNP rs109007595 (SEQ. ID. NO. 10) is C/T, the predefined value V assigned to this SNP is comprised between 103 and 106, preferably about 105;
if the genotype at the gene locus corresponding to the SNP rs109578101 (SEQ. ID. NO. 11) is C/T, the predefined value V assigned to this SNP is comprised between 104 and 106, preferably about 106;
if the genotype at the gene locus corresponding to the SNP rs137182814 (SEQ. ID. NO. 12) is C/G, the predefined value V assigned to this SNP is comprised between 103 and 106, preferably about 105;
if the genotype at the gene locus corresponding to said SNP rs109975461 (SEQ. ID. NO. 32) is C/T, the predefined value V assigned to this SNP is comprised between 98 and 101, preferably about 99;
if the genotype at the gene locus corresponding to said SNP rs134589272 (SEQ. ID. NO. 33) is C/T, the predefined value V assigned to this SNP is comprised between 98 and 101, preferably about 99.

Preferably, if the genotype at the gene locus corresponding to the SNP rs43703012 (SEQ. ID. NO. 2) is A/A, the predefined value V for this SNP with SEQ. ID. NO. 2 is not assigned and does not contribute to the determination of the IAC.

Advantageously, the applicants have found that the correlation between the IAC calculated according to the method of the invention and the IAC obtained according to standard methods increases if, during step a) of said method of the invention, the presence or absence is determined in the biological sample of the bovine animal of other polymorphisms in addition to those indicated above with SEQ. ID. NO. 1 to 12, 32 and 33.

In particular, this correlation and therefore the accuracy of the method of the invention is increased if, during said step a), in addition to at least three of the previously indicated polymorphisms the genotype is also determined in the biological sample of the bovine animal at the gene locus corresponding to at least three homozygous or heterozygous single nucleotide polymorphisms SNP selected from among:
SNP rs110454169 (SEQ. ID. NO. 13) of the Glycerol-3-phosphate acyltransferase 6 (AGPAT6) gene;
SNP rs109686238 (SEQ. ID. NO. 14) of the C—C motif chemokine 3 (CCL3) gene;
SNP rs135514413 (SEQ. ID. NO. 15) of the Protein C-ets-2 (ETS2) gene;
SNP rs109231659 variant 1 (SEQ. ID. NO. 16) of the Growth hormone receptor (GHR) gene;
SNP rs109231659 variant 3 (SEQ. ID. NO. 17) of the Growth hormone receptor (GHR) gene;
SNP rs136905033 (SEQ. ID. NO. 18) of the Phosphatidate phosphatase lipin 1 (LPIN1) gene;
SNP rs110590698 (SEQ. ID. NO. 19) of the Lipoprotein lipase (LPL) gene;
SNP rs43765461 (SEQ. ID. NO. 20) of the Lactotransferrin (LTF) gene;
SNP rs41257077 (SEQ. ID. NO. 21) of the Protease inhibitor 2 (PI) gene;
SNP rs110270855 (SEQ. ID. NO. 22) of the Phospholipase C-β 1 (PLCB1) gene;
SNP rs133669403 (ID. SEQ. NO. 23) of the PPARG coactivator 1-α (PPARGC1A) gene;
SNP rs110684599 (SEQ. ID. NO. 24) of the Prolactin (PRL) gene;
SNP rs43706906 (SEQ. ID. NO. 25) of the Signal transducer and activator of transcription 1-α/β (STAT1) gene.

Preferably, the method provides for determining the genotype at the gene locus corresponding to at least six polymorphisms selected from those with SEQ. ID. NO. 13 to 25.

More preferably, the method of the invention provides for determining the genotype at the gene locus corresponding to all thirteen polymorphisms denoted above with SEQ. ID. NO. 13 to 25.

The method of the invention further provides, during step b), to assign a predefined value V also to polymorphisms determined in step a) among those with SEQ. ID. NO. 13 to 25, as well as assigning this predefined value also to determined SNPs with SEQ. ID. NO. 1 to 12 as indicated above.

The predefined value V to be assigned to such SNPs is comprised between:
98 and 101 for SNP rs110454169 (SEQ. ID. NO. 13);
97 and 100 for SNP rs109686238 (SEQ. ID. NO. 14);
89 and 93 for SNP rs135514413 (SEQ. ID. NO. 15);
96 and 104 for SNP rs109231659 variant 1 (SEQ. ID. NO. 16);
95 and 106 for SNP rs109231659 variant 3 (SEQ. ID. NO. 17);
97 and 103 for SNP rs136905033 (SEQ. ID. NO. 18);
104 and 108 for SNP rs110590698 (SEQ. ID. NO. 19);
98 and 103 for SNP rs43765461 (SEQ. ID. NO. 20);
97 and 102 for SNP rs41257077 (SEQ. ID. NO. 21);
96 and 104 for SNP rs110270855 (SEQ. ID. NO. 22);
93 and 107 for SNP rs133669403 (SEQ. ID. NO. 23);
98 and 100 for SNP rs110684599 (SEQ. ID. NO. 24);
98 and 103 for SNP rs43706906 (SEQ. ID. NO. 25).

Further, the method of the invention provides, during said step c), for calculating the arithmetic mean of the predefined values V assigned to all the polymorphisms determined in step a), i.e., the arithmetic mean of the predefined values V assigned to the SNPs determined among those with SEQ. ID. NO. 1 to 12, 32 and 33 and the predefined values V assigned to the SNPs determined from those with SEQ. ID. NO. 13 to 25.

The arithmetic mean obtained essentially corresponds to the IAC of the milk to be produced by the bovine animal under consideration.

According to an aspect of the method of the invention, following determination in the biological sample of the bovine animal of the genotype at the gene locus corresponding to at least three of the homozygous or heterozygous single nucleotide polymorphisms having SEQ. ID. NO. 13 to 25, the above method requires the predefined value V to be comprised between:
98 and 100, preferably about 99, if the genotype at the gene locus corresponding to the SNP rs110454169 (SEQ. ID. NO. 13) is C/C or TT;
98 and 100, preferably about 99, if the genotype at the gene locus corresponding to the SNP rs109686238 (SEQ. ID. NO. 14) is C/C or T/T;
89 and 93, preferably about 91, if the genotype at the gene locus corresponding to the SNP rs135514413 (SEQ. ID. NO. 15) is A/A;
97 and 102, preferably about 99, if the genotype at the gene locus corresponding to the SNP rs109231659 variant 1 (SEQ. ID. NO. 16) is G/G or T/T;
95 and 106, preferably about 100, if the genotype at the gene locus corresponding to the SNP rs109231659 variant 3 (SEQ. ID. NO. 17) is G/G or T/T;
97 and 103, preferably about 100, if the genotype at the gene locus corresponding to the SNP rs136905033 (SEQ. ID. NO. 18) is C/C or T/T;
104 and 108, preferably about 106, if the genotype at the gene locus corresponding to the SNP rs110590698 (SEQ. ID. NO. 19) is A/A;
98 and 103, preferably about 100, if the genotype at the gene locus corresponding to the SNP rs43765461 (SEQ. ID. NO. 20) is C/C or T/T;
97 and 102, preferably about 100, if the genotype at the gene locus corresponding to the SNP rs41257077 (SEQ. ID. NO. 21) is C/C or T/T;
96 and 104, preferably about 100, if the genotype at the gene locus corresponding to the SNP rs110270855 (SEQ. ID. NO. 22) is C/C or T/T;
93 and 107, preferably about 100, if the genotype at the gene locus corresponding to the SNP rs133669403 (SEQ. ID. NO. 23) is A/A or G/G;
98 and 100, preferably about 99, if the genotype at the gene locus corresponding to the SNP rs110684599 (SEQ. ID. NO. 24) is A/A or C/C;
98 and 103, preferably about 101, if the genotype at the gene locus corresponding to the SNP rs43706906 (SEQ. ID. NO. 25) is C/C or G/G.

Preferably, also:
the predefined value V is comprised between 98 and 100, preferably about 99, if the genotype at the gene locus corresponding to the SNP rs110454169 (SEQ. ID. NO. 13) is C/T;
the predefined value V is comprised between 98 and 100, preferably about 99, if the genotype at the gene locus corresponding to the SNP rs109686238 (SEQ. ID. NO. 14) is C/T;
the predefined value V is comprised between 96 and 104, preferably about 100, if the genotype at the gene locus corresponding to the SNP rs109231659 variant 1 (SEQ. ID. NO. 16) is G/T;
the predefined value V is comprised between 95 and 105, preferably about 100, if the genotype at the gene locus corresponding to the SNP rs109231659 variant 3 (SEQ. ID. NO. 17) is G/T;
the predefined value V is comprised between 98 and 102, preferably about 100, if the genotype at the gene locus corresponding to the SNP rs136905033 (SEQ. ID. NO. 18) is C/T;
the predefined value V is comprised between 98 and 102, preferably about 100, if the genotype at the gene locus corresponding to the SNP rs43765461 (SEQ. ID. NO. 20) is C/T;
the predefined value V is comprised between 98 and 101, preferably about 100, if the genotype at the gene locus corresponding to the SNP rs41257077 (SEQ. ID. NO. 21) is C/T;
the predefined value V is comprised between 98 and 101, preferably about 100, if the genotype at the gene locus corresponding to the SNP rs110270855 (SEQ. ID. NO. 22) is C/T;
the predefined value V is comprised between 99 and 102, preferably about 101, if the genotype at the gene locus corresponding to the SNP rs133669403 (SEQ. ID. NO. 23) is A/G;
the predefined value V is comprised between 98 and 100, preferably about 99, if the genotype at the gene locus corresponding to the SNP rs110684599 (SEQ. ID. NO. 24) is A/C;
the predefined value V is comprised between 98 and 102, preferably about 101, if the genotype at the gene locus corresponding to the SNP rs43706906 (SEQ. ID. NO. 25) is C/G.

Still preferably, if the genotype at the gene locus corresponding to the SNP rs135514413 (SEQ. ID. NO. 15) is A/C or C/C the predefined value V for this SNP with SEQ. ID. NO. 15 is not assigned and therefore does not contribute to the determination of the IAC.

Further preferably, if the genotype at the gene locus corresponding to the SNP rs110590698 (SEQ. ID. NO. 19) is T/T or A/T the predefined value V for this SNP with SEQ. ID. NO. 19 is not assigned and therefore does not contribute to the determination of the IAC.

As can be seen from the following examples, the determination of the genotype of the biological sample at the SNPs with SEQ. ID. NO. 1 to 12, 32 and 33 combined with the genotype determination at the SNPs with SEQ. ID. NO. 13 to 25 allows to obtain a correlation of up to 0.7 between the value of the IAC calculated by the method of the invention and the value of IAC identified by standard methods.

It is not excluded that, according to a variant of the method of the invention, step a) does not include the determination of polymorphisms selected from those having SEQ. ID. NO. 13 to 25.

According to another aspect of the method of the invention, this method provides for determining in the biological sample of the bovine animal, during said step a), in addition to at least fourteen polymorphisms selected from those having SEQ. ID. NO. 1 to 12, 32 and 33 and, optionally, at least three polymorphisms selected from those having SEQ. ID. NO. 13 to 25, also the genotype at the gene locus corresponding to at least three homozygous or heterozygous single nucleotide polymorphisms selected from:
SNP rs43706475 (SEQ. ID. NO. 26) of the Casein kappa (CSN3) gene;
SNP rs109231659 variant 2 (SEQ. ID. NO. 27) of the Growth Hormone Receptor (GHR) gene;
SNP rs109579682 (SEQ. ID. NO. 28) of the PPARG coactivator 1-α (PPARGC1A) gene;
SNP rs109428015 (SEQ. ID. NO. 29) of the Prolactin receptor (PRLR) gene.

Preferably, the method also provides for determining the genotype at the gene locus corresponding to at least one homozygous or heterozygous single nucleotide polymorphism selected from:
SNP rs211156498 (SEQ. ID. NO. 30) of the Casein alpha-S2 (CS1N1S2) gene;
SNP rs465820291 (SEQ. ID. NO. 31) of the Diacylglycerol O-acyltransferase 1 (DGAT1) gene.

More preferably, the method of the invention provides for determining the genotype at the gene locus corresponding to at least six polymorphisms selected from those with SEQ. ID. NO. 26 to 31.

Even more preferably, the method of the invention provides for determining the genotype at the gene locus corresponding to all six polymorphisms indicated above with SEQ. ID. NO. 26 to 31.

According to this aspect of the method of the invention, it further provides, during step b), to assign a predefined value V also to the polymorphisms determined in step a) among those with SEQ. ID. NO. 26 to 33, as well as assigning this predefined value also to the determined SNPs with SEQ. ID. NO. 1 to 12, 32 and 33, preferably with SEQ. ID. NO. 1 to 25, as indicated above.

The predefined value V to be assigned to such SNPs is comprised between:
96 and 101 for SNP rs43706475 (SEQ. ID. NO. 26);
93 and 98 for SNP rs109231659 variant 2 (SEQ. ID. NO. 27);
99 and 103 for SNP rs109579682 (SEQ. ID. NO. 28);
82 and 116 for SNP rs109428015 (SEQ. ID. NO. 29);
100 and 105 for SNP rs211156498 (SEQ. ID. NO. 30);
90 and 111 for SNP rs465820291 (SEQ. ID. NO. 31).

Further, the method of the invention, during step c), provides for calculating the arithmetic mean of the predefined values V assigned to all the polymorphisms determined in step a), i.e., the arithmetic mean of the predefined values V assigned to the SNPs determined from those with SEQ. ID. NO. 1 to 12, 32 and 33, preferably SEQ. ID. NO. 1 to 25, 32 and 33 and of the predefined values V assigned to the SNPs determined from those with SEQ. ID. NO. 26 to 31.

The arithmetic mean obtained essentially corresponds to the IAC of the milk that will be produced by the genotyped bovine animal.

According to one aspect of the method of the invention, the predefined value V is comprised between:
96 and 101, preferably about 98, if the genotype at the gene locus corresponding to the SNP rs43706475 (SEQ. ID. NO. 26) is T/T;
93 and 98, preferably about 96, if the genotype at the gene locus corresponding to the SNP rs109231659 variant 2 (SEQ. ID. NO. 27) is T/T;
99 and 101, preferably about 100, if the genotype at the gene locus corresponding to the SNP rs109579682 (SEQ. ID. NO. 28) is C/C or T/T;
88 and 110, preferably about 99, if the genotype at the gene locus corresponding to the SNP rs109428015 (SEQ. ID. NO. 29) is C/C or T/T;
100 and 105, preferably about 103, if the genotype at the gene locus corresponding to the SNP rs211156498 (SEQ. ID. NO. 30) is A/A;
93 and 107, preferably about 100, if the genotype at the gene locus corresponding to the SNP rs465820291 (SEQ. ID. NO. 31) is C/C or G/G.

Preferably, also:
the predefined value V is comprised between 98 and 102, preferably about 101, if the genotype at the gene locus corresponding to the SNP rs109579682 (SEQ. ID. NO. 28) is C/T;
the predefined value V is comprised between 101 and 103, preferably about 102, if the genotype at the gene locus corresponding to the SNP rs109428015 (SEQ. ID. NO. 29) is C/T;
the predefined value V is comprised between 99 and 104, preferably about 102, if the genotype at the gene locus corresponding to the SNP rs465820291 (SEQ. ID. NO. 31) is C/G.

Still preferably, if the genotype at the gene locus corresponding to the SNP rs43706475 (SEQ. ID. NO. 26) is G/T or G/G, the predefined value V for this SNP with SEQ. ID. NO. 26 is not assigned and therefore does not contribute to the determination of the IAC.

In addition, preferably, if the genotype at the gene locus corresponding to the SNP rs109231659 variant 2 (SEQ. ID. NO. 27) is A/T or A/A, the predefined value V for this SNP with SEQ. ID. NO. 27 is not assigned and therefore does not contribute to the determination of the IAC.

Further preferably, if the genotype at the gene locus corresponding to the SNP rs211156498 (SEQ. ID. NO. 30) is A/G or G/G, the predefined value V for this SNP with SEQ. ID. NO. 30 is not assigned and therefore does not contribute to the determination of the IAC.

It is not excluded that, according to a variant of the method of the invention, the only polymorphisms determined during step a) are selected from those having SEQ. ID. NO. 1 to 12, 32 and 33 and SEQ. ID. NO. 26 to 31, thus excluding the determination of polymorphisms having SEQ. ID. NO. 13 to 25.

It is not excluded that, according to another variant of the method of the invention, step a) does not include the determination of polymorphisms selected from those having SEQ. ID. NO. 26 to 31.

The method of the present invention is preferably realisable by means of a gene chip, which will be described below.

It is specified that the operations necessary for the extraction and handling of a nucleic acid from a biological sample of a bovine animal and the identification of the presence/absence of predefined SNPs in said nucleic acid and, further, any operations necessary for the correct use of a gene chip for the identification of nucleotide sequences represent knowledge known in the art, which will therefore not be further discussed in this document.

A gene chip, in particular an SNP gene chip for identifying the milk clotting ability index (IAC) of milk produced by a bovine animal, is therefore also part of the present invention.

The chip of the present invention is a DNA array.

It should be noted that the terms gene chip, chip, SNP chip, microarray, gene chip, biochip or DNA chip are to be considered synonymous.

The chip of the present invention makes it possible to simultaneously analyse the activity of a plurality of genes related to the milk clotting ability of milk.

In general, it is noted that the possibility of applying nucleotide sequences for the construction of chips for the analysis of molecular markers of the SNP type varies according to the biochemical technology used, as each developer has its own different biochemical platform for microarray development. This means that the results obtained with different chips but with the same sequences locked onto the chip, as known in literature, are not comparable. For the chip of the invention, at a preliminary stage the identified SNPs were tested on different microarrays to assess the actual efficiency of the final genotyping result and select the most accurate one. Preferably, the chip of the invention is manufactured using Illumina Infinium array technology®.

According to the preferred embodiment of the chip of the invention, it comprises, as probes locked onto the surface of such chip, at least fourteen nucleotide sequences different from each other, particularly DNA nucleotide sequences, selected from nucleotide sequences having at least 90% identity with the fourteen SEQ. ID. NO. 1 to 12, 32 and 33.

It is specified that, in this document, “percentage identity” between two DNA nucleotide sequences means the percentage of identical nucleotide residues at corresponding positions in the two optimally aligned compared sequences.

To determine this percentage identity value, the two sequences are aligned with each other and, optionally, interruptions can be introduced, such as deletions or insertions (also arranged at the ends of the sequences). Nucleotide residues in corresponding positions are then compared. When a position in the first sequence is occupied by the same nucleotide residue that occupies the corresponding position in the second sequence, the molecules are considered to be identical at that position. The percentage identity between two sequences therefore depends on the number of identical positions shared by the compared sequences, and basically corresponds to [(number of identical positions/total number of positions)×100].

The percentage identity can also be obtained using mathematical algorithms of a known type.

The term “corresponding position” refers to a position in a sequence of a nucleotide that corresponds (faces), following alignment, to a certain position in a reference sequence.

Returning to the preferred embodiment of the chip of the invention, preferably, said probes are locked in a known position on the surface of a support to form a microgrid such that, during the use of said chip, it is possible to identify each of said probes uniquely.

This support may include, for example, a slide.

The chip according to the preferred embodiment of the invention is configured to simultaneously identify the presence or absence in the biological sample of the bovine animal of each genotype at the gene locus corresponding to at least fourteen homozygous or heterozygous single nucleotide polymorphisms SNP included in the group of the SNPs presenting SEQ. ID. NO. 1 to 12, 32 and 33, as defined above.

More preferably, the chip of the invention comprises, as probes locked onto the surface of such chip, at least fourteen nucleotide sequences different from each other, selected from the twelve nucleotide sequences having SEQ. ID. NO. 1 to 12, 32 and 33.

Optionally, each of these probes consists of a single-helix DNA segment corresponding to each of these sequences.

Optionally, each probe may be locked onto the chip in one or more copies at the same site.

Advantageously, the greater the number of locked copies, the greater the signal strength on the chip.

Optionally, the gene chip of the present invention may also comprise positive or negative control probes.

These control probes are selected from probes known in the art for verifying the correct use of a chip.

By way of non-limiting example, negative control probes may include probes from organisms not belonging to the animal class of interest, mammals.

Advantageously, the gene chip of the invention is configured to allow the determination of SNP polymorphisms in the biological sample during the aforementioned step a) of the method for predicting the IAC described above.

According to the preferred embodiment of the gene chip of the invention, it further comprises, as probes locked onto the surface of the chip, at least three nucleotide sequences different from each other, particularly DNA nucleotide sequences, selected from nucleotide sequences having at least 90% identity with the thirteen SEQ. ID. NO. 13 to 25.

Preferably, such a chip comprises at least six nucleotide sequences different from each other, selected from among the nucleotide sequences having at least 90% identity with the thirteen SEQ. ID. NO. 13 to 25.

More preferably, the chip comprises at least thirteen nucleotide sequences different from each other, selected from among the nucleotide sequences having at least 90% identity with the thirteen SEQ. ID. NO. 13 to 25.

Even more preferably, the chip of the invention comprises, as probes locked onto the surface of such chip, at least three, preferably at least six, nucleotide sequences different from each other, selected from the thirteen nucleotide sequences having SEQ. ID. NO. 13 to 25.

Preferably, the chip of the present invention comprises, as probes locked onto the surface of such chip, at least the thirteen nucleotide sequences having SEQ. ID. NO. 13 to 25.

It is not excluded that, according to an alternative embodiment of the chip of the invention, it does not comprise, as probes locked onto the surface, nucleotide sequences having at least 90% identity with SEQ. ID. NO. 13 to 25.

According to the preferred embodiment of the SNP chip of the invention, it further comprises, as probes locked onto the surface of the gene chip, at least three nucleotide sequences different from each other, particularly DNA nucleotide sequences, selected from nucleotide sequences having at least 90% identity with the six SEQ. ID. NO. 26 to 31.

Preferably, the chip comprises at least six nucleotide sequences different from each other, selected from among the nucleotide sequences having at least 90% identity with the six SEQ. ID. NO. 26 to 31.

Even more preferably, the chip of the present invention comprises, as probes locked onto the surface of such chip, at least three, preferably at least six, nucleotide sequences different from each other, selected from the six nucleotide sequences having SEQ. ID. NO. 26 to 31.

Preferably, the chip of the present invention comprises, as probes locked onto the surface of such chip, at least the six nucleotide sequences having SEQ. ID. NO. 26 to 33.

It is not excluded that, according to a variant of the chip of the invention, it comprises, as probes locked onto the surface, nucleotide sequences selected from the nucleotide sequences having at least 90% identity with SEQ. ID. NO. 1 to 12, 32 and 33 and with SEQ. ID. NO. 26 to 31, and does not include nucleotide sequences having at least 90% identity with SEQ. ID. NO. 13 to 25.

It is also not excluded that, according to another variant of the chip of the invention, it does not comprise, as probes locked onto the surface, nucleotide sequences having at least 90% identity with SEQ. ID. NO. 26 to 31.

The examples below are given for the purpose of a better understanding of what is described and are not intended as limitations of the claims.

### Examples

### 1.1 Calculation of the IAC According to the Standard Method

The milk clotting ability index was calculated in a standard way on milk samples from 4719 bovine animals.

In particular, the following phenotypic characteristics of the milk tested were determined by means of a lactodynamograph:
(I) r: expresses the time (in minutes) elapsing from the addition of rennet until the start of the milk coagulation process;
(II) a30: expresses the consistency of the curd in millimetres measured 30 minutes after the addition of rennet;
(III) acidity [pH];
(IV) concentration of saturated, unsaturated, monounsaturated, polysaturated fatty acids;
(V) concentration of ketosis indicators such as acetone and beta-hydroxybutyrate.

For the standard determination of the IAC, these characteristics, in particular r and a30, were related according to the following formula:

IAC=100+[((a30-averageₐ₃₀)/dSₐ₃₀)*2.5−((r-averageᵣ)/dsᵣ)*2.5]
where ds is the standard deviation.

### 1.2. Calculation of the IAC by the Invention

The IAC was calculated by the method of the invention, on the same animals used in example 1.1.

In particular, DNA of each bovine animal was extracted from biological samples of hair, skin, semen or nasal mucus for each of these animals using DNeasy® Blood & Tissue Kits (QIAGEN® Group) according to the instructions provided by the supplier.

For each genotyped animal, the IAC derived from the analysis of the results obtained from the determination of three groups of SNP polymorphisms, defined as follows, was calculated using the method of the invention:
cla1=determination in the bovine animal of the SNPs with SEQ. ID. NO. 1 to 12;
cla2=determination in the bovine animal of the SNPs with SEQ. ID. NO. 13 to 25;
cla3=determination in the bovine animal of the SNPs with SEQ. ID. NO. 26 to 33;
cla4=determination in the bovine animal of the SNPs with SEQ. ID. NO. 1 to 12, 32 and 33.

### 1.3 Correlation Between the IAC Obtained in Example 1.1 and the IAC Obtained in Example 1.2

The values obtained in examples 1.1 and 1.2 were correlated with each other to assess the correlation by computation of the data with Rstudio (standard method based on phenotypes Vs method of the invention based on genotypes).

Table 1 shows the correlations between the standard IAC and the IAC obtained according to the invention by determination of the SNPs cla1, cla2 and cla3, ranging from 0.186 to 0.242 for the IACs obtained according to the invention.

TABLE 1

| Gender of | Number of | IAC | IAC | IAC |
|---|---|---|---|---|
| animals | animals | (cla1) | (cla2) | (cla3) |
| Males + | 4719 | 0.197 | 0.230 | 0.235 |
| females | | | | |
| Males | 758 | 0.139 | 0.186 | 0.207 |
| Females | 3961 | 0.210 | 0.240 | 0.242 |

In addition, the correlation between the standard IAC and the three IACs was calculated as a function of the reliability class of the standard IAC. The correlation values are shown in Table 2 and range from 0.128 to 0.

TABLE 2

| IAC | Number of | IAC | IAC | IAC |
|---|---|---|---|---|
| reliability | animals | (cla1) | (cla2) | (cla3) |
| >50 | 3981 | 0.218 | 0.250 | 0.254 |
| >60 | 669 | 0.144 | 0.212 | 0.236 |
| >70 | 534 | 0.159 | 0.226 | 0.256 |
| >75 | 451 | 0.160 | 0.235 | 0.261 |
| >80 | 330 | 0.128 | 0.251 | 0.277 |
| >85 | 205 | 0.207 | 0.316 | 0.343 |
| >90 | 125 | 0.278 | 0.434 | 0.423 |
| >95 | 63 | 0.335 | 0.485 | 0.500 |

Subsequently, only male animals were considered, divided into two classes: with a reliability of less than 70% and with a reliability of 70% or more.

The correlation values obtained are shown in Table 3.

TABLE 3

| IAC | Number of | IAC | IAC | IAC |
|---|---|---|---|---|
| reliability | animals | (cla1) | (cla2) | (cla3) |
| <70 | 533 | 0.094 | 0.077 | 0.074 |
| ≥70 | 225 | 0.159 | 0.224 | 0.254 |

Finally, the IAC obtained according to the method of the invention was related to the EBV (Estimated Breeding Value) calculated from the phenotypic data of the animals genotyped using the PEST algorithm.

The correlations are shown in Table 4 and have been broken down by reliability rates.

TABLE 4

| IAC | Number of | IAC | IAC | IAC |
|---|---|---|---|---|
| reliability | animals | (cla1) | (cla2) | (cla1 + 2 + 3) |
| >0 | 3743 | 0.239 | 0.238 | 0.243 |
| >50 | 2898 | 0.253 | 0.263 | 0.267 |
| >60 | 547 | 0.138 | 0.168 | 0.168 |
| >70 | 427 | 0.171 | 0.179 | 0.177 |
| >80 | 221 | 0.247 | 0.259 | 0.223 |
| >90 | 74 | 0.481 | 0.443 | 0.350 |
| >95 | 34 | 0.414 | 0.517 | 0.359 |
| >98 | 10 | 0.554 | 0.709 | 0.191 |

Furthermore, the IAC obtained by the determination of the cla4 SNPs in accordance with embodiments of the present invention was related to the EBV (Estimated Breeding Value) calculated from phenotypic data of the genotyped animals using the PEST algorithm. The correlations for the cla4 SNP are shown in Table 5 where they have been broken down by reliability rates, and also compared with the corresponding correlations for the cla1 SNPs.

TABLE 5

| IAC | Number of | IAC | IAC |
|---|---|---|---|
| reliability | animals | (cla1) | (cla4) |
| >50 | 2898 | 0.253 | 0.270 |
| >60 | 547 | 0.138 | 0.170 |

These data show that the use of the cla4 SNPs leads to an improvement in the calculation accuracy ranging from 2%, with >50 reliability, to 3%, with >60 reliability.

### Listing of Sequences

In the attached DNA nucleotide sequences, nucleotides are indicated by their standard code corresponding to a letter (A, T, C, G).

Polymorphisms and their position in the sequences are represented using the following codes corresponding to a letter: R represents the polymorphism [G/A]; Y represents the polymorphism [T/C]; M represents the polymorphism [A/C]; K represents the polymorphism [G/T]; S represents the polymorphism [G/C]; W represents the polymorphism [A/T], all in accordance with the “Annex C, Appendix 2” of “STANDARD ST.25-STANDARD FOR THE PRESENTATION OF NUCLEOTIDE AND AMINO ACID SEQUENCE LISTINGS IN PATENT APPLICATIONS”.

Where more than one polymorphism is present in the sequence, the polymorphism of interest for the purposes of the present invention is represented by the letter identifying the polymorphism described in the present description to the corresponding sequence number.

Finally, it is specified that for:
SNP rs109231659 variant 1 (SEQ. ID. NO. 16), the [G/T] polymorphism of interest in the present invention is indicated by the letter K in position 151;
SNP rs133669403 (SEQ. ID. NO. 23), the [A/G] polymorphism of interest in the present invention is indicated by the letter R in position 151.

### REFERENCES

De Marchi M. et al., Prediction of coagulation properties, titratable acidity, and pH of bovine milk using mid-infrared spectroscopy, J. Dairy Sci., 2009, 92:423-432;
Pecorari M. et al., Attitudine alla coagulazione dei latti delle razze Frisona, Bruna, Reggiana e Modenese. Sci. Tecn. Latt.-cas., 1987, 38, 376.

1. A gene chip for predicting the milk clotting ability index (IAC) of the milk produced by a bovine animal, the gene chip comprising, as probes locked onto the surface of said gene chip, at least fourteen DNA nucleotide sequences different from each other selected from among the DNA nucleotide sequences having at least 90% identity with the fourteen SEQ. ID. NO. 1 to 12, 32 and 33; said gene chip being configured to simultaneously identify the presence or absence in a biological sample of said bovine animal of each genotype at the gene locus also corresponding to at least fourteen homozygous or heterozygous single nucleotide polymorphisms (SNP) comprised in the group of polymorphisms (SNP) constituted by:
SNP rs109913786 (SEQ. ID. NO. 1) of the Glycerol-3-phosphate acyltransferase 6 (AGPAT6) gene;
SNP rs43703012 (SEQ. ID. NO. 2) of the Casein β (CSN2) gene;
SNP rs43703015 (SEQ. ID. NO. 3) of the Casein kappa (CSN3) gene;
SNP rs43703017 (SEQ. ID. NO. 4) of the Casein kappa (CSN3) gene;
SNP rs110137537 (SEQ. ID. NO. 5) of the Hormone-sensitive lipase (LIPE) gene;
SNP rs43765462 (SEQ. ID. NO. 6) of the Lactotransferrin (LTF) gene;
SNP rs134390757 (SEQ. ID. NO. 7) of the LXR-a oxysterol receptor (LXRα) gene;
SNP rs135588030 (SEQ. ID. NO. 8) of the Oxidized low-density lipoprotein receptor 1 (ORL1) gene;
SNP rs41624917 (SEQ. ID. NO. 9) of the Phospholipase C epsilon 1 (PLCE1) gene;
SNP rs109007595 (SEQ. ID. NO. 10) of the POU class 1 homeobox 1 (POU1F1) gene;
SNP rs109578101 (SEQ. ID. NO. 11) of the Signal transducer and activator of transcription 5A (STAT5A) gene;
SNP rs137182814 (SEQ. ID. NO. 12) of the Signal transducer and activator of transcription 5A (STAT5A) gene;
SNP rs109975461 (SEQ. ID. NO. 32) of the Cholinergic receptor nicotinic epsilon subunit (CHRNE) gene;
SNP rs134589272 (SEQ. ID. NO. 33) of the Scribble cell polarity complex component LLGL2 (FDXR LLGL2) gene.

2. The gene chip according to claim 1, further comprising, as probes locked on the surface of said gene chip, at least three DNA nucleotide sequences different from each other selected from among the DNA nucleotide sequences having at least 90% identity with the thirteen SEQ. ID. NO. 13 to 25; said gene chip being configured to simultaneously identify the presence or absence in said biological sample of said bovine animal of each genotype at the gene locus also corresponding to at least three homozygous or heterozygous single nucleotide polymorphisms (SNP) comprised in the group of polymorphisms (SNP) constituted by:
SNP rs110454169 (SEQ. ID. NO. 13) of the Glycerol-3-phosphate acyltransferase 6 (AGPAT6) gene;
SNP rs109686238 (SEQ. ID. NO. 14) of the C—C 3 motif chemokine (CCL3) gene;
SNP rs135514413 (SEQ. ID. NO. 15) of the Protein C-ets-2 (ETS2) gene;
SNP rs109231659 variant 1 (SEQ. ID. NO. 16) of the Growth hormone receptor (GHR) gene;
SNP rs109231659 variant 3 (SEQ. ID. NO. 17) of the Growth hormone receptor (GHR) gene;
SNP rs136905033 (SEQ. ID. NO. 18) of the Phosphatidate phosphatase lipin-1 (LPIN1) gene;
SNP rs110590698 (SEQ. ID. NO. 19) of the Lipoprotein lipase (LPL) gene;
SNP rs43765461 (SEQ. ID. NO. 20) of the Lactotransferrin (LTF) gene;
SNP rs41257077 (SEQ. ID. NO. 21) of the Protease inhibitor 2 (PI) gene;
SNP rs110270855 (SEQ. ID. NO. 22) of the Phospholipase C-β 1 (PLCB1) gene;
SNP rs133669403 (SEQ. ID. NO. 23) of the PPARG coactivator 1-α (PPARGC1A) gene;
SNP rs110684599 (SEQ. ID. NO. 24) of the Prolactin (PRL) gene;
SNP rs43706906 (SEQ. ID. NO. 25) of the Signal transducer and activator of transcription 1-α/β (STAT1) gene.

3. The gene chip according to claim 1, further comprising, as probes locked on the surface of said gene chip, at least three DNA nucleotide sequences different from each other, selected from the DNA nucleotide sequences having at least 90% identity with the six SEQ. ID. NO. 26 to 31; said gene chip being configured to simultaneously identify the presence or absence in said biological sample of said bovine animal of each genotype at the gene locus also corresponding to at least three single homozygous or heterozygous nucleotide polymorphisms (SNP) included in the group of polymorphisms (SNP) constituted by:
SNP rs43706475 (SEQ. ID. NO. 26) of the Casein kappa (CSN3) gene;
SNP rs109231659 variant 2 (SEQ. ID. NO. 27) of the Growth hormone receptor (GHR) gene;
SNP rs109579682 (SEQ. ID. NO. 28) of the PPARG coactivator 1-a (PPARGC1A) gene;
SNP rs109428015 (SEQ. ID. NO. 29) of the Prolactin receptor (PRLR) gene;
SNP rs211156498 (SEQ. ID. NO. 30) of the Casein alpha-S2 (CS1N1S2) gene;
SNP rs465820291 (SEQ. ID. NO. 31) of the Diacylglycerol O-acyltransferase 1 (DGAT1) gene.

4. A method for predicting the milk clotting ability index (IAC) of the milk produced by a bovine animal, comprising the following steps:
a) determining in a biological sample of said bovine animal the genotype at the gene locus corresponding to at least fourteen homozygous or heterozygous single nucleotide polymorphisms (SNP) selected from the group of polymorphisms (SNP) having SEQ. ID. NO. 1 to 12, 32 and 33, as defined in claim 1;
b) assigning a predefined value (V) to each of the polymorphisms (SNP, SEQ. ID. NO. 1-12, 32 and 33) determined in step a), said predefined value (V) being comprised between:
102 and 106 for SNP rs109913786 (SEQ. ID. NO. 1);
86 and 125 for SNP rs43703012 (SEQ. ID. NO. 2);
100 and 110 for SNP rs43703015 (SEQ. ID. NO. 3);
103 and 108 for SNP rs43703017 (SEQ. ID. NO. 4);
102 and 107 for SNP rs110137537 (SEQ. ID. NO. 5);
102 and 108 for SNP rs43765462 (SEQ. ID. NO. 6);
102 and 107 for SNP rs134390757 (SEQ. ID. NO. 7);
105 and 107 for SNP rs135588030 (SEQ. ID. NO. 8);
103 and 106 for SNP rs41624917 (SEQ. ID. NO. 9);
100 and 109 for SNP rs109007595 (SEQ. ID. NO. 10);
102 and 109 for SNP rs109578101 (SEQ. ID. NO. 11);
103 and 107 for SNP rs137182814 (SEQ. ID. NO. 12);
98 and 101 for SNP rs109975461 (SEQ. ID. NO. 32);
98 and 101 for SNP rs134589272 (SEQ. ID. NO. 33);
c) calculating the arithmetic mean of the predefined values (V) assigned in step b) to the polymorphisms (SNP, SEQ. ID. NO. 1-12, 32 and 33) determined in step a), said arithmetic mean substantially corresponding to said milk clotting ability index (IAC).

5. The method according to claim 4, wherein
if the genotype at the gene locus corresponding to said SNP rs109913786 (SEQ. ID. NO. 1) is C/C or T/T, the predefined value (V) assigned to said SNP rs109913786 (SEQ. ID. NO. 1) is comprised between 103 and 106;
if the genotype at the gene locus corresponding to said SNP rs43703012 (SEQ. ID. NO. 2) is C/C, the predefined value (V) assigned to said SNP rs43703012 (SEQ. ID. NO. 2) is comprised between 99 and 102;
if the genotype at the gene locus corresponding to said SNP rs43703015 (SEQ. ID. NO. 3) is C/C or T/T, the predefined value (V) assigned to said SNP rs43703015 (SEQ. ID. NO. 3) is comprised between 102 and 109;
if the genotype at the gene locus corresponding to said SNP rs43703017 (SEQ. ID. NO. 4) is A/A or G/G, the predefined value (V) assigned to said SNP rs43703017 (SEQ. ID. NO. 4) is comprised between 104 and 106;
if the genotype at the gene locus corresponding to said SNP rs110137537 (SEQ. ID. NO. 5) is A/A or C/C, the predefined value (V) assigned to said SNP rs110137537 (SEQ. ID. NO. 5) is comprised between 103 and 106;
if the genotype at the gene locus corresponding to said SNP rs43765462 (SEQ. ID. NO. 6) is G/G or T/T, the predefined value (V) assigned to said SNP rs43765462 (SEQ. ID. NO. 6) is comprised between 103 and 107;
if the genotype at the gene locus corresponding to said SNP rs134390757 (SEQ. ID. NO. 7) is C/C or T/T, the predefined value (V) assigned to said SNP rs134390757 (SEQ. ID. NO. 7) is comprised between 103 and 106;
if the genotype at the gene locus corresponding to said SNP rs135588030 (SEQ. ID. NO. 8) is A/A or G/G, the predefined value (V) assigned to said SNP rs135588030 (SEQ. ID. NO. 8) is comprised between 105 and 107;
if the genotype at the gene locus corresponding to said SNP rs41624917 (SEQ. ID. NO. 9) is C/C or T/T, the predefined value (V) assigned to said SNP rs41624917 (SEQ. ID. NO. 9) is comprised between 103 and 105;
if the genotype at the gene locus corresponding to said SNP rs109007595 (SEQ. ID. NO. 10) is C/C or T/T, the predefined value (V) assigned to said SNP rs109007595 (SEQ. ID. NO. 10) is comprised between 102 and 107;
if the genotype at the gene locus corresponding to said SNP rs109578101 (SEQ. ID. NO. 11) is C/C or T/T, the predefined value (V) assigned to said SNP rs109578101 (SEQ. ID. NO. 11) is comprised between 103 and 108;
if the genotype at the gene locus corresponding to said SNP rs137182814 (SEQ. ID. NO. 12) is C/G or G/G, the predefined value (V) assigned to said SNP rs137182814 (SEQ. ID. NO. 12) is comprised between 104 and 106;
if the genotype at the gene locus corresponding to said SNP rs109975461 (SEQ. ID. NO. 32) is C/C or T/T, the predefined value (V) assigned to said SNP rs109975461 (SEQ. ID. NO. 32) is comprised between 99 and 101;
if the genotype at the gene locus corresponding to said SNP rs134589272 (SEQ. ID. NO. 33) is C/C or T/T, the predefined value (V) assigned to said SNP rs134589272 (SEQ. ID. NO. 33) is comprised between 98 and 101.

6. The method according to claim 4, wherein
if the genotype at the gene locus corresponding to said SNP rs109913786 (SEQ. ID. NO. 1) is C/T, the predefined value (V) assigned to said SNP rs109913786 (SEQ. ID. NO. 1) is comprised between 103 and 105;
if the genotype at the gene locus corresponding to said SNP rs43703012 (SEQ. ID. NO. 2) is A/C, the predefined value (V) assigned to said SNP rs43703012 (SEQ. ID. NO. 2) is comprised between 108 and 111;
if the genotype at the gene locus corresponding to said SNP rs43703015 (SEQ. ID. NO. 3) is C/T, the predefined value (V) assigned to said SNP rs43703015 (SEQ. ID. NO. 3) is comprised between 104 and 106;
if the genotype at the gene locus corresponding to said SNP rs43703017 (SEQ. ID. NO. 4) is A/G, the predefined value (V) assigned to said SNP rs43703017 (SEQ. ID. NO. 4) is comprised between 104 and 107;
if the genotype at the gene locus corresponding to said SNP rs110137537 (SEQ. ID. NO. 5) is A/C, the predefined value (V) assigned to said SNP rs110137537 (SEQ. ID. NO. 5) is comprised between 104 and 106;
if the genotype at the gene locus corresponding to said SNP rs43765462 (SEQ. ID. NO. 6) is G/T, the predefined value (V) assigned to said SNP rs43765462 (SEQ. ID. NO. 6) is comprised between 104 and 106;
if the genotype at the gene locus corresponding to said SNP rs134390757 (SEQ. ID. NO. 7) is C/T, the predefined value (V) assigned to said SNP rs134390757 (SEQ. ID. NO. 7) is comprised between 103 and 105;
if the genotype at the gene locus corresponding to said SNP rs135588030 (SEQ. ID. NO. 8) is A/G, the predefined value (V) assigned to said SNP rs135588030 (SEQ. ID. NO. 8) is comprised between 105 and 107;
if the genotype at the gene locus corresponding to said SNP rs41624917 (SEQ. ID. NO. 9) is C/T, the predefined value (V) assigned to said SNP rs41624917 (SEQ. ID. NO. 9) is comprised between 103 and 105;
if the genotype at the gene locus corresponding to said SNP rs109007595 (SEQ. ID. NO. 10) is C/T, the predefined value (V) assigned to said SNP rs109007595 (SEQ. ID. NO. 10) is comprised between 103 and 106;
if the genotype at the gene locus corresponding to said SNP rs109578101 (SEQ. ID. NO. 11) is C/T, the predefined value (V) assigned to said SNP rs109578101 (SEQ. ID. NO. 11) is comprised between 104 and 106;
if the genotype at the gene locus corresponding to said SNP rs137182814 (SEQ. ID. NO. 12) is C/G, the predefined value (V) assigned to said SNP rs137182814 (SEQ. ID. NO. 12) is comprised between 103 and 106;
if the genotype at the gene locus corresponding to said SNP rs109975461 (SEQ. ID. NO. 32) is C/T, the predefined value (V) assigned to said SNP rs109975461 (SEQ. ID. NO. 32) is comprised between 98 and 101;
if the genotype at the gene locus corresponding to said SNP rs134589272 (SEQ. ID. NO. 33) is C/T, the predefined value (V) assigned to said SNP rs134589272 (SEQ. ID. NO. 33) is comprised between 98 and 101.

7. The method according to claim 4, wherein said biological sample comprises a nucleic acid of said bovine animal, said determination of the genotype at the gene locus corresponding to said at least three polymorphisms (SNP) being performed through the analysis of said nucleic acid.

8. The method according to claim 4, wherein the method it determines, during said step a), in said biological sample of said bovine animal also the genotype at the gene locus corresponding to at least three homozygous or heterozygous single nucleotide polymorphisms (SNP) selected from the group of the SNPs having SEQ. ID. NO. 13 to 25, as follows:
SNP rs110454169 (SEQ. ID. NO. 13) of the Glycerol-3-phosphate acyltransferase 6 (AGPAT6) gene;
SNP rs 109686238 (SEQ. ID. NO. 14) of the C—C 3 motif chemokine (CCL3) gene;
SNP rs135514413 (SEQ. ID. NO. 15) of the Protein C-ets-2 (ETS2) gene;
SNP rs109231659 variant 1 (SEQ. ID. NO. 16) of the Growth hormone receptor (GHR) gene;
SNP rs109231659 variant 3 (SEQ. ID. NO. 17) of the Growth hormone receptor (GHR) gene;
SNP rs136905033 (SEQ. ID. NO. 18) of the Phosphatidate phosphatase lipin-1 (LPIN1) gene;
SNP rs110590698 (SEQ. ID. NO. 19) of the Lipoprotein lipase (LPL) gene;
SNP rs43765461 (SEQ. ID. NO. 20) of the Lactotransferrin (LTF) gene;
SNP rs41257077 (SEQ. ID. NO. 21) of the Protease inhibitor 2 (PI) gene;
SNP rs110270855 (SEQ. ID. NO. 22) of the Phospholipase C-1 (PLCB1) gene;
SNP rs133669403 (SEQ. ID. NO. 23) of the PPARG coactivator 1-α (PPARGC1A) gene;
SNP rs110684599 (SEQ. ID. NO. 24) of the Prolactin (PRL) gene;
SNP rs43706906 (SEQ. ID. NO. 25) of the Signal transducer and activator of transcription 1-α/β (STAT1) gene;
said method further providing, during said step b), for assigning a predefined value (V) to said polymorphisms (SNP, SEQ. ID. NO. 13-25) determined during said step a), said predefined value (V) being comprised between:
98 and 100, if the genotype at the gene locus corresponding to said SNP rs110454169 (SEQ. ID. NO. 13) is C/C or T/T, said predefined value (V) being comprised between 98 and 100, if the genotype at the gene locus corresponding to said SNP rs110454169 (SEQ. ID. NO. 13) is C/T;
98 and 100, if the genotype at the gene locus corresponding to said SNP rs 109686238 (SEQ. ID. NO. 14) is C/C or T/T, said predefined value (V) being comprised between 98 and 100, if the genotype at the gene locus corresponding to said SNP rs109686238 (SEQ. ID. NO. 14) is C/T;
89 and 93, if the genotype at the gene locus corresponding to said SNP rs135514413 (SEQ. ID. NO. 15) is A/A;
97 and 102, if the genotype at the gene locus corresponding to said SNP rs109231659 variant 1 (SEQ. ID. NO. 16) is G/G or T/T, said predefined value (V) being comprised between 96 and 104, if the genotype at the gene locus corresponding to said SNP rs109231659 variant 1 (SEQ. ID. NO. 16) is G/T;
95 and 106, if the genotype at the gene locus corresponding to said SNP rs109231659 variant 3 (SEQ. ID. NO. 17) is G/G or T/T, said predefined value (V) being comprised between 95 and 105, if the genotype at the gene locus corresponding to said SNP rs109231659 variant 3 (SEQ. ID. NO. 17) is G/T;
97 and 103, if the genotype at the gene locus corresponding to said SNP rs136905033 (SEQ. ID. NO. 18) is C/C or T/T, said predefined value (V) being comprised between 98 and 102, if the genotype at the gene locus corresponding to said SNP rs136905033 (SEQ. ID. NO. 18) is C/T;
104 and 108, if the genotype at the gene locus corresponding to said SNP rs110590698 (SEQ. ID. NO. 19) is A/A;
98 and 103, if the genotype at the gene locus corresponding to said SNP rs43765461 (SEQ. ID. NO. 20) is C/C or T/T, said predefined value (V) being comprised between 98 and 102, if the genotype at the gene locus corresponding to said SNP rs43765461 (SEQ. ID. NO. 20) is C/T;
97 and 102, if the genotype at the gene locus corresponding to said SNP rs41257077 (SEQ. ID. NO. 21) is C/C or T/T, said predefined value (V) being comprised between 98 and 101, if the genotype at the gene locus corresponding to said SNP rs41257077 (SEQ. ID. NO. 21) is C/T;
96 and 104, if the genotype at the gene locus corresponding to said SNP rs110270855 (SEQ. ID. NO. 22) is C/C or T/T, said predefined value (V) being comprised between 98 and 101, if the genotype at the gene locus corresponding to said SNP rs110270855 (SEQ. ID. NO. 22) is C/T;
93 and 107, if the genotype at the gene locus corresponding to said SNP rs133669403 (SEQ. ID. NO. 23) is A/A or G/G, said predefined value (V) being comprised between 99 and 102, if the genotype at the gene locus corresponding to said SNP rs133669403 (SEQ. ID. NO. 23) is A/G;
98 and 100, if the genotype at the gene locus corresponding to said SNP rs110684599 (SEQ. ID. NO. 24) is A/A or C/C, said predefined value (V) being comprised between 98 and 100, preferably about 99, if the genotype at the gene locus corresponding to said SNP rs110684599 (SEQ. ID. NO. 24) is A/C;
98 and 103, if the genotype at the gene locus corresponding to said SNP rs43706906 (SEQ. ID. NO. 25) is C/C or G/G, said predefined value (V) being comprised between 98 and 102, if the genotype at the gene locus corresponding to said SNP rs43706906 (SEQ. ID. NO. 25) is C/G;
said method further providing, in said step c), for calculating the arithmetic mean of the predefined values (V) assigned to all the polymorphisms (SNP, SEQ. ID. NO. 1-25, 32 and 33) determined in said step a), said arithmetic mean substantially corresponding to said milk clotting ability index (IAC).

9. The method according to claim 4, wherein the method determines, during said step a), in said biological sample of said bovine animal, also the genotype at the gene locus corresponding to at least three homozygous or heterozygous single nucleotide polymorphisms (SNP) selected from the group of the SNPs having SEQ. ID. NO. 26 to 31, as follows:
SNP rs43706475 (SEQ. ID. NO. 26) of the Casein kappa (CSN3) gene;
SNP rs109231659 variant 2 (SEQ. ID. NO. 27) of the Growth hormone receptor (GHR) gene;
SNP rs109579682 (SEQ. ID. NO. 28) of the PPARG coactivator 1-a (PPARGC1A) gene;
SNP rs109428015 (SEQ. ID. NO. 29) of the Prolactin receptor (PRLR) gene;
SNP rs211156498 (SEQ. ID. NO. 30) of the Casein alpha-S2 (CS1N1S2) gene;
SNP rs465820291 (SEQ. ID. NO. 31) of the Diacylglycerol O-acyltransferase 1 (DGAT1) gene;
said method further providing, during said step b), for assigning a predefined value (V) also to said polymorphisms (SNP, SEQ. ID. NO. 26-31) determined during said step a), said predefined value (V) being comprised between:
96 and 101, if the genotype at the gene locus corresponding to said SNP rs43706475 (SEQ. ID. NO. 26) is T/T;
93 and 98, if the genotype at the gene locus corresponding to said SNP rs 109231659 variant 2 (SEQ. ID. NO. 27) is T/T;
99 and 101, if the genotype at the gene locus corresponding to said SNP rs 109579682 (SEQ. ID. NO. 28) is C/C or T/T, said predefined value (V) being comprised between 98 and 102, if the genotype at the gene locus corresponding to said SNP rs109579682 (SEQ. ID. NO. 28) is C/T;
88 and 110, if the genotype at the gene locus corresponding to said SNP rs109428015 (SEQ. ID. NO. 29) is C/C or T/T, said predefined value (V) being comprised between 101 and 103, if the genotype at the gene locus corresponding to said SNP rs109428015 (SEQ. ID. NO. 29) is C/T;
100 and 105, if the genotype at the gene locus corresponding to said SNP rs211156498 (SEQ. ID. NO. 30) is A/A;
93 and 107, if the genotype at the gene locus corresponding to said SNP rs465820291 (SEQ. ID. NO. 31) is C/C or G/G, said predefined value (V) being comprised between 99 and 104, preferably about 102, if the genotype at the gene locus corresponding to said SNP rs465820291 (SEQ. ID. NO. 31) is C/G;
said method further providing, in said step c), for calculating the arithmetic mean of the predefined values (V) assigned to all the polymorphisms (SNP, SEQ. ID. NO. 1-33) determined in said step a), said arithmetic mean substantially corresponding to said milk clotting ability index (IAC).

10. The method according to claim 4, wherein during said step a), said determination in said biological sample of the genotype at the gene locus corresponding to said polymorphisms (SNP) is performed by means of a gene chip comprising, as probes locked onto the surface of said gene chip, at least fourteen DNA nucleotide sequences different from each other selected from among the DNA nucleotide sequences having at least 90% identity with the fourteen SEQ. ID. NO. 1 to 12, 32 and 33; said gene chip being configured to simultaneously identify the presence or absence in a biological sample of said bovine animal of each genotype at the gene locus also corresponding to at least fourteen homozygous or heterozygous single nucleotide polymorphisms (SNP) comprised in the group of polymorphisms (SNP) constituted by:
SNP rs109913786 (SEQ. ID. NO. 1) of the Glycerol-3-phosphate acyltransferase 6 (AGPAT6) gene;
SNP rs43703012 (SEQ. ID. NO. 2) of the Casein β (CSN2) gene;
SNP rs43703015 (SEQ. ID. NO. 3) of the Casein kappa (CSN3) gene;
SNP rs43703017 (SEQ. ID. NO. 4) of the Casein kappa (CSN3) gene;
SNP rs110137537 (SEQ. ID. NO. 5) of the Hormone-sensitive lipase (LIPE) gene;
SNP rs43765462 (SEQ. ID. NO. 6) of the Lactotransferrin (LTF) gene;
SNP rs134390757 (SEQ. ID. NO. 7) of the LXR-a oxysterol receptor (LXRα) gene;
SNP rs135588030 (SEQ. ID. NO. 8) of the Oxidized low-density lipoprotein receptor 1 (ORL1) gene;
SNP rs41624917 (SEQ. ID. NO. 9) of the Phospholipase C epsilon 1 (PLCE1) gene;
SNP rs 109007595 (SEQ. ID. NO. 10) of the POU class 1 homeobox 1 (POU1F1) gene;
SNP rs109578101 (SEQ. ID. NO. 11) of the Signal transducer and activator of transcription 5A (STAT5A) gene;
SNP rs137182814 (SEQ. ID. NO. 12) of the Signal transducer and activator of transcription 5A (STAT5A) gene;
SNP rs109975461 (SEQ. ID. NO. 32) of the Cholinergic receptor nicotinic epsilon subunit (CHRNE) gene;
SNP rs134589272 (SEQ. ID. NO. 33) of the Scribble cell polarity complex component LLGL2 (FDXR LLGL2) gene.

11. The method according to claim 7, wherein said biological sample is a sample of fur, skin, semen or nasal mucus of said bovine animal.
